(19) United States
(12) Patent Application Publication
(10) Pub. No.: US 2024/0091440 A1
(43) Pub. Date: Mar. 21, 2024

(54) DRUG DELIVERY DEVICE AND SYSTEM

(71) Applicant: AMGEN INC., Thousand Oaks, CA (US)

(72) Inventors: Scott R. Gibson, Thousand Oaks, CA (US); Mehran Mojarrad, Thousand Oaks, CA (US); Paul D. Faucher, Escondido, CA (US); Antonio S. Murcia, Oceanside, CA (US); Alan D. Payne, Escondido, CA (US); Sheldon Moberg, Thousand Oaks, CA (US); Olivia Li, Simi Valley, CA (US)

(21) Appl. No.: 17/767,469

(22) PCT Filed: Oct. 16, 2020
(86) PCT No.: PCT/US20/55874

§ 371 Date: Apr. 8, 2022

Related U.S. Application Data
(60) Provisional application No. 62/925,676, filed on Oct. 24, 2019.
(60) Provisional application No. 62/924,087, filed on Oct. 21, 2019.
(60) Provisional application No. 62/923,367, filed on Oct. 18, 2019.

(51) Int. Cl. A61M 5/172; A61M 5/142; A61M 39/10

(52) U.S. Cl. CPC A61M 5/1723; A61M 5/14244; A61M 39/10; A61M 2205/3331

(57) ABSTRACT

A drug delivery device for delivering a medicament includes a housing, a pump, drive component, an inlet fluid path, an outlet fluid path, an inlet pressure sensor, an outlet pressure sensor, and a controller. The pump is coupled with the housing. The drive component is for driving the pump. The inlet fluid path is configured to deliver medicament to the pump. The outlet fluid path is configured to receive medicament from the pump. The inlet pressure sensor is positioned along the inlet fluid path and configured to measure inlet fluid pressure. The outlet pressure sensor is positioned along the outlet fluid path and configured to measure outlet fluid pressure. The controller is workingly coupled with the inlet pressure sensor, the outlet pressure sensor, and the drive component, wherein the controller is configured to adjust at least one parameter of the drive component based on input information received from the inlet pressure sensor and/or the outlet pressure sensor.

### CROSS-REFERENCE TO RELATED APPLICATIONS

Priority is claimed to U.S. Provisional Patent Application No. 62/923,367, filed Oct. 18, 2019, and to U.S. Provisional Patent Application No. 62/924,087, filed Oct. 21, 2019, and to U.S. Provisional Patent Application No. 62/925,676, filed Oct. 24, 2019, and the entire contents of each of the foregoing are hereby incorporated herein by reference.

### FIELD OF THE DISCLOSURE

The present disclosure generally relates to drug delivery devices and systems and, more particularly, to a pump and a system for long-term, continuous, semi-continuous, and/or intravenous drug delivery.

### BACKGROUND

Drugs are administered to treat a variety of conditions and diseases. Intravenous (“IV”) therapy is a drug dosing process that delivers drugs directly into a patient's vein using an infusion contained in a delivery container such as IV bag and tubing connected to a needle subsystem that fluidically communicates with the reservoir through the pump assembly collectively called infusion set. These drug dosings may be performed in a healthcare facility, or in some instances, at remote locations such as a patient's home. In certain applications, a drug delivery process may last for an extended period of time (e.g., for one hour or longer) or may include continuous or semi-continuous delivery of a drug over an extended period of time (e.g., for several hours, days, weeks, or longer). For many of these relatively long-term delivery requirements, a pump is often utilized to control and/or administer the drug to the patient. The pump may be coupled (physically, fluidly, and/or otherwise) to various components, such as a drug delivery container, supply lines, connection ports, and/or the patient.

It may be desirable to utilize a pump and/or overall system that is portable and/or wearable. It may also be desirable to utilize a pump and an overall system that minimizes patient inconvenience, minimizes the size and profile of the device and the overall system, minimizes the complexity of the device and overall system, minimizes the noise and vibration of the device, accommodates easy connection/disconnection and changeover of the infusion set, simplifies or automates priming of the line, accommodate easy delivery interruption and reestablishment based on required therapy and delivery profile, easily provides status of delivery and other important user information such as occlusion and volume of drug delivered or remaining in the reservoir, reduces the cost of the device and the overall system, increases the reliability and accuracy of the device and the overall system.

As described in more detail below, the present disclosure sets forth systems and methods for drug delivery embodying advantageous alternatives to existing systems and methods, and that may address one or more of the challenges or needs mentioned herein, as well as provide other benefits and advantages.

### BRIEF DESCRIPTION OF THE DRAWINGS

The above needs are at least partially met through provision of the systems and approaches for drug delivery device reconstitution described in the following detailed description, particularly when studied in conjunction with the drawings, wherein:

FIG. 1 illustrates an exemplary drug delivery device in accordance with various embodiments;

FIG. 2 illustrates a partial cross-section of an exemplary drug delivery device in accordance with various embodiments;

FIG. 3 illustrates an exploded view of an exemplary drug delivery device in accordance with various embodiments;

FIG. 4 illustrates an exploded view of an exemplary drive assembly for a drug delivery device in accordance with various embodiments;

FIG. 5 illustrates an exploded view of an exemplary pump head for a drug delivery device in accordance with various embodiments;

FIG. 6 illustrates an exploded view of an exemplary pressure sensor assembly and manifold assembly for a drug delivery device in accordance with various embodiments;

FIG. 7 illustrates an exploded view of an exemplary PCA and battery assembly for a drug delivery device in accordance with various embodiments;

FIG. 8 is a flow chart for an exemplary controller for a drug delivery device in accordance with various embodiments;

FIG. 9 illustrates an exemplary drug delivery system in accordance with various embodiments;

FIG. 10 illustrates a view of an exemplary pressure sensor in accordance with various embodiments;

FIG. 11 illustrates a view of an exemplary drug delivery device in accordance with various embodiments;

FIG. 12 illustrates a view of an exemplary drug delivery device in accordance with various embodiments;

FIG. 13 illustrates a view of an exemplary drug delivery device in a non-attached configuration, in accordance with various embodiments;

FIG. 14 illustrates a view of an exemplary drug delivery device in a non-attached and an attached configuration, in accordance with various embodiments;

FIG. 15 illustrates a view of an exemplary drug delivery device in a non-attached and an attached configuration, in accordance with various embodiments;

FIG. 16 illustrates a view of an exemplary drug delivery device in a non-attached and an attached configuration, in accordance with various embodiments;

FIGS. 17 and 18 illustrate an exemplary drug delivery device in accordance with various embodiments;

FIGS. 19-21 each illustrates an exemplary drug delivery device pressure sensor and tubing in accordance with various embodiments;

FIGS. 22-23 each illustrates an exemplary drug delivery device pump head and tubing in accordance with various embodiments;

FIG. 24 illustrates an exemplary drug delivery device in accordance with various embodiments;

FIG. 25 illustrates a partial cross-section of an exemplary drug delivery device in accordance with various embodiments;

FIG. 26 illustrates an exemplary drug delivery device and system in accordance with various embodiments; and

FIGS. 27A and 27B illustrate two exemplary drug delivery devices in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### GENERAL DESCRIPTION

The present disclosure relates to drug delivery devices and systems. In some aspects the present disclosure relates to a drug delivery system with a pump and a fluid path for long-term, continuous, semi-continuous, and/or intravenous drug delivery. Under some conditions, a drug delivery process may last for an extended period of time (e.g., for one hour or longer) or may include continuous or semi-continuous delivery of a drug over an extended period of time (e.g., for several hours, days, weeks, or longer) or may include delivery via an intravenous connection to a patient. The present disclosure utilizes various features for potentially improved drug dose accuracy and/or improved pump controls, while maintaining a relatively compact sized system that may be desirable or appropriate for extended, continuous, semi-continuous, and/or intravenous delivery.

For example, the present disclosure includes a drug delivery device for delivering a medicament, having a housing; a pump coupled with the housing; a drive component for driving the pump; an inlet fluid path configured to deliver medicament to the pump; an outlet fluid path configured to receive medicament from the pump; an inlet pressure sensor positioned along the inlet fluid path and configured to measure inlet fluid pressure; an outlet pressure sensor positioned along the outlet fluid path and configured to measure outlet fluid pressure; and a controller workingly coupled with the inlet pressure sensor, the outlet pressure sensor, and the drive component. The controller may be configured to adjust at least one parameter of the drive component based on input information received from the inlet pressure sensor and/or the outlet pressure sensor. For example, the controller may be utilized and/or able to detect an occlusion event based on the input information received from the inlet pressure sensor and/or the outlet pressure sensor. Additionally or alternatively, the controller may be utilized and/or able to detect a low medicament event based on the input information received from the inlet pressure sensor and/or the outlet pressure sensor.

The drug delivery device may be utilized in a drug delivery system, including a medicament container containing a medicament; an inlet fluid path configured to receive the medicament from the medicament container; and an outlet fluid path configured to deliver the medicament to a patient. The drug delivery system may also include an adaptor for fluidly connecting at least two sections of the inlet fluid path with each other.

The drug delivery device may include a controller configured to deliver the medicament at an accuracy rate of at least 95%. As a more specific example, the controller may include an encoder-fed, closed loop system. As an even more specific example, the drug delivery device may further include an encoder board for determining measured drive speed and a motor model for determining a calculated drive speed. The accuracy rate may be measured in a variety of ways, such as:
by comparing the amount of volume of drug product that the pump was programmed to delivered and the amount of volume actually delivered;
by comparing the amount of weight of drug product that the pump was programmed to delivered and the amount of weight actually delivered;
by comparing the amount of volume of drug product that the pump controls have calculated should have been delivered and the amount of volume of drug product actually delivered;
by comparing the amount of weight of drug product that the pump controls have calculated should have been delivered and the amount of weight of drug product actually delivered;
by comparing the programmed total time of delivery and the actual total time of delivery; or
other suitable methods.
By monitoring discrete quantities of fluid delivered per cycle of peristalisis or motor rotation and adjust for any variability due to air entrapment in line or other causes of drift in flow rate

In other aspects, the present disclosure includes a drug delivery device for delivering a medicament, having a housing; a fluid displacement assembly at least partially supported by and/or surrounded by the housing, the fluid displacement assembly including a ring tube portion; a drive component at least partially supported by and/or surrounded by the housing, the drive component including an eccentric component having a contact surface configured to directly or indirectly compress the ring tube portion a compression distance such that when the eccentric component rotates about an axis, the contact surface moves along generally circular path and drives the medicament through the fluid displacement assembly; and a compression sensor workingly coupled with the eccentric component and configured to measure input relating to and/or proportional to the compression distance.

The device may also include a controller workingly coupled with the compression sensor and the drive component, wherein the controller is configured to adjust at least one parameter of the drive component based on input information received from the compression sensor. For example, the drug device may further include an inlet fluid path configured to deliver medicament to the fluid displacement assembly; an outlet fluid path configured to receive medicament from the fluid displacement assembly; an inlet pressure sensor positioned along the inlet fluid path and configured to measure inlet fluid pressure; an outlet pressure sensor positioned along the outlet fluid path and configured to measure outlet fluid pressure; and a controller workingly coupled with the inlet pressure sensor, the outlet pressure sensor, the compression sensor, and the drive component, wherein the controller is configured to adjust at least one parameter of the drive component based on input information received from the inlet pressure sensor, the outlet pressure sensor and/or the compression sensor. The compression sensor may be an optical sensor, a resistance sensor, or any other suitable sensor.

Aspects of the present disclosure may be utilized for improving dose accuracy of peristaltic pump. For example, sensor(s) may be utilized to measure ring tube compression to potentially improve pump displacement accuracy. As an example, peristaltic pumps such as ring pumps displace fluid by compressing a portion of a tube and then moving along generally circular path and to urge the fluid forward. Generally speaking, and up to a certain point, the more completely a tube is compressed, the more liquid will be urged through the tube. Therefore, variances in tube stiffness and other properties may affect accuracy and/or predictability of pump displacement volume. The present disclosure includes components, devices, and methods for measuring pump compression and utilizing the same, such as a feedback-loop to a pump controller to increase the likelihood of uniform flowrate regardless of tubing physical properties, thereby potentially improving dose accuracy and overall pump performance.

Current peristaltic pumps used for IV infusion of drugs such as oncology products often use silicone, polyvinyl chloride (PVC), ethyl vinyl acetate (EVA), polyolefin, cyclic polyolefin polymer (COP), cyclic polyolefin copolymer or Tygon type clear tubing that are relatively soft but may have different physical properties such as stiffness and modulus of elasticity. The variation between physical properties can influence performance and/or accuracy of the peristaltic pump if there is no sensor to adjust for the required pressure to squeeze the tube uniformly per cycle of pump rotation.

The present disclosure utilizes various in-line sensors in a closed-loop feedback system to adjust for rotation rate or applied pressure in order to maintain a desired flow rate per specific therapy in mind. For example, to deliver oncolytic compound such as Blincyto® over 7-10 days from a 250 mL IV bag reservoir when using a peristaltic pump, a uniform flow rate can be calculated and programmed into the infusion pump. However, such a calculation may not account for variations in material properties to compensate for the pressure applied on the tubing to achieve the intended flowrate of the medication. As an example, if a ring pump is used as the primary drive module for the pump, the flexible/rigid ring applies a defined pressure on the flexible tubing at each cycle, sometimes without feedback on the actual displacement of the tube per cycle of rotation of the pump rotor. However, providing feedback, such as tube displacement information, may provide feedback for improving pump accuracy and overall performance.

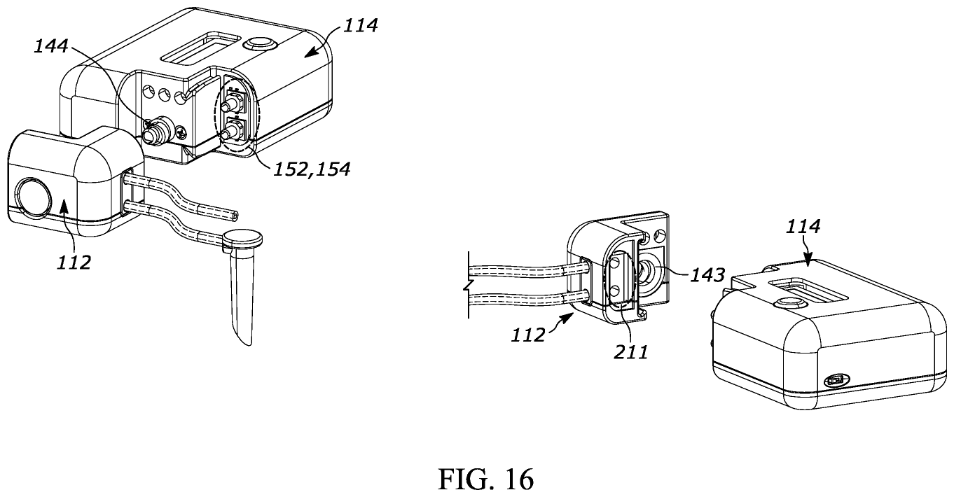

In one such method, utilizing an optical-based sensor, a light source (e.g., LED, OLED, etc.) is positioned on one side of a tube while a complementary metal-oxide-semiconductor (CMOS) detector elements are placed in the opposite side to register the amount of light transmitted. As the tube is squeezed by the pressure element, such as a ring or other flexible spring-like elements at the pump head, the inner diameter is distorted changing the transmission and refractive indices of the tube. This inner diameter displacement can be related to volume of fluid being dispensed per cycle of rotation of the rotor element (as will be discussed in more detail below with respect to FIG. 16). The controller then may be able to adjust the speed of rotation of the rotor in real-time to achieve the desired flow rate. The position of the emitter and detector is preferably determined based on the overall configuration of the pump-head. For example, the emitter LED could be above the tube while the detector array element can be fitted into the ring spring assembly within the pump-head. Other forms of interfaces such as rollers could also be used for peristaltic actuation.

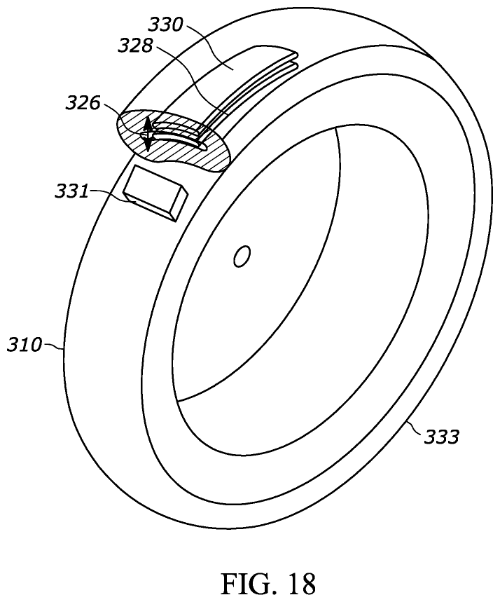

In another such method, utilizing a resistance-based sensor, electrode elements may be imbedded into a segment of the tubing in the inner diameter of the tube. When the tube is fully squeezed so that inner diameter is fully collapsed and the two electrodes touch, a short circuit can be established to close a circuit as the trigger to indicate sufficient pressure applied to the tube to achieve the desired flowrate (as will be discussed in more detail below with respect to FIG. 18). In this configuration, an active ring utilizing actuator(s) such as PZT element(s) or any other form known to the art may be used to modulate the pressure applied on the tube to achieve full collapse during rotation of the pump-head rotor.

In still other aspects, the present disclosure includes a drug delivery system for delivering a medicament to a user, comprising: a medicament container containing a medicament; a fluid path configured to at least selectively fluidly connected the medicament container and the user; a sensor positioned adjacent and/or along the fluid path and configured to determine an interruption in the fluid connection between the medicament container and the user; and a drug delivery device positioned adjacent to and/or along the fluid path. The drug delivery device may include a housing; a pump coupled with the housing; a drive component for driving the medicament through the pump; and a controller workingly coupled with the drive component, wherein the controller is configured to adjust at least one parameter of the drive component based on input information received from the sensor.

The controller may be programmed to selectively discontinue operation of the pump based on input information received from the sensor. The sensor may be positioned adjacent to and/or within a connector that us used to disconnect (fluidly and/or mechanically) the medicament container and the user.

The sensor may be a flow sensor. Additionally or alternatively, the sensor may be a transmitter-based sensor, a circuit-based sensor, a magnet-based sensor, or another suitable sensor.

Intravenous infusion pump systems are generally comprised of several key component devices. Typically, these include a pump, a drug reservoir, and an infusion set (such as an IV tube) for fluidly connecting the drug reservoir with the patient. The infusion set may contain an in-line filter to prevent air ingress to the vein. Additionally, the infusion set may have one portion which includes an access needle for insertion into the vein, and another portion which includes a needle, luer type fitting or other fluid connection to the reservoir. When delivering drug, these infusion set parts act as a single fluid path connecting the reservoir to the patient's vein. These portions are typically connected using a fluid tight couple such as a luer fitting and may include a valve or other couple member to prevent flow and to sustain cleanliness of the fluid path when the couple is disconnected. The pump may be adjacent to and/or along the fluid path to urge the fluid flow into the patient's body at a desired flow rate and/or duration.

For example, when on therapy, delivery can be continuous through a day or days or weeks without many interruptions (if any) except during change of drug reservoir. Delivery may continue through normal daily activities. Delivery may continue through dressing, eating, bathing, walking, driving, travel, sleeping and all activities. To patients, the weight and bulk of the pump may interfere with these activities. Pumps which are not generally water-proof must be kept out of the spray of water while showering. Some users are known to hang their pumps outside the shower while the infusion set is still inserted in their vein, and while drug continues to be delivered. Pumps which are generally heavy and bulky interfere with quality rest and sleep. Pumps which are generally heavy and bulky and which include infusion sets may limit the ability to interact with young children or grandchildren. Infusion lines may catch on furniture or other objects as patients walk or move past.

What is desired is a system which allows, within prescribed limits, patients to disconnect from their delivery system safely, which recognizes the disconnection provides alert(s) to the patient and/or other members of the patient's health community to ensure a safe outcome. It is also beneficial to have components and/or methods to detect such a disconnection, whether the disconnection has been intentional or unintentional. This disclosure addresses several embodiments for detecting a fluid path disconnection.

For example, the disconnection sensor may be adjacent to and/or within a fluid path connector. As a more specific embodiment, one embodiment uses a sensor or sensors placed at the fluid connector in such a way that when the connector is disconnected a signal is generated and, in turn, detected by the pump. For example, the signal may be an electrical signal, a radio signal, or any other suitable type of signal. Alternately, the signal may indicate the fluid couple is connected and the absence of signal may indicate the couple is disconnected.

In one such example, the sensor may be or utilize a transmitter which changes state when the fluid connector is disconnected. For example, the sensor may be held in a low power or “off” state when the fluid couple is connected and then move to a high power or “on” state and transmit when the fluid couple is disconnected. Such a transmission may be radio waves and may provide a detected signal at the pump which itself contains a radio receiver. Such a system might use Bluetooth, Bluetooth low energy, zigby/mesh network, wifi, or similar protocol.

In another such example, the sensor may utilize an electrical circuit which originates and terminates at the pump but which travels through or along the length or a partial length of the infusion set and optionally the reservoir between origin and terminal. Such a system may detect an electrical signal received at the terminal point in the pump. For example, when the fluid couple is connected, the conductive path is complete, but when disconnected the fluid couple is broken and no signal reaches the terminal point in the circuit. The electrical signal may be through a switch or an electrical contact set to complete the circuit directly.

Alternately, the sensor may utilize a magnetically driven circuit. For example, the sensor may leverage field detection technologies such as magnetic resistors, giant magnetic resistors, reed switch or other solid-state equivalents and/or a magnet of suitable field strength. The magnetic driven circuit may be configured to utilize a coupling detection circuit.

These concepts would likely take advantage of a displacement between two halves of the fluid path to change the detected field strength. Detected field strength is known to be proportional to the distance between the field source and the point of detection. For example, one half of the fluid coupling could contain a field detector and the second half could contain a magnet of suitable field strength to drive a threshold field level when the state of coupling is changed. This can be made to complete an electrical switch and therefore a sense circuit. This can also be configured to output a low power signal when the couple is connected or when the couple is disconnected (changing the strength of the signal when the connection is changed). Either configuration has advantages. For example, having a low power state when coupled would enable the use of minimal power during most of the useful life reduces the required battery size and makes the device smaller. However, using a higher power state which suppresses the alert when coupled would enable a “fail safe” alert to user if power were lost due to circuit or certain power failures. Another configuration of magnet and detector could have both on the same half of the fluid couple, perhaps preferably proximal to the pump, but could use the action of coupling or disconnecting to displace the magnet relative to the detector (possible graphic of a spring-loaded ring containing the magnet which is displaced curing coupling).

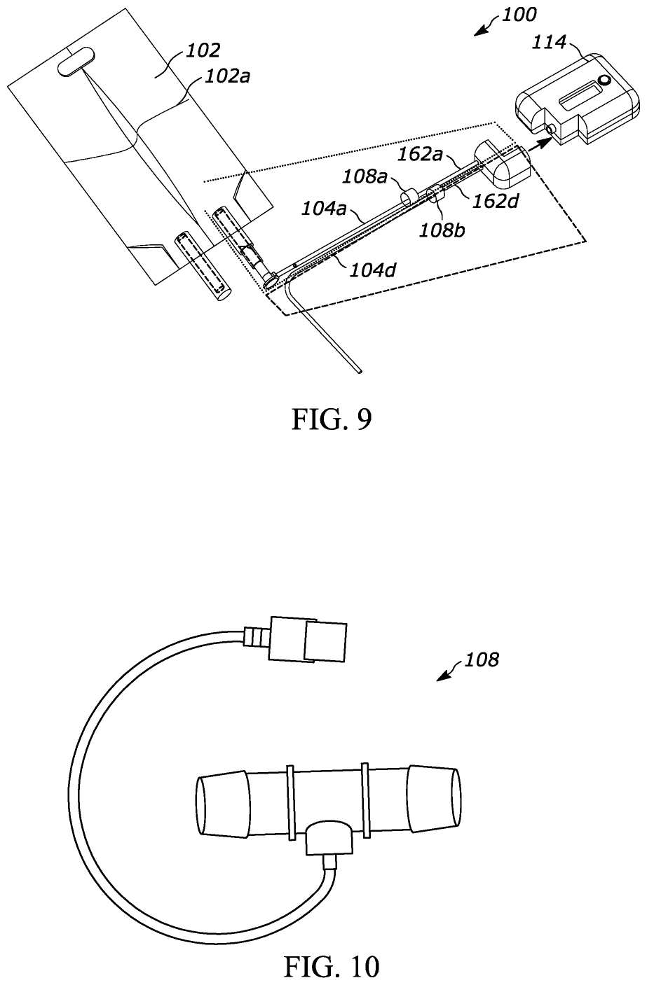

Alternatively or additionally, MEMS-based sensors may be incorporated into or around the tubing sets. These sensors are generally robust through the gamma irradiation sterilization process so they may be utilized even in applications that require external sterilization. These sensors include (1) a fluid path portion that receives connectors and/or tubing and (2) electronic components that include external wiring that runs to a receiver (e.g., FIG. 9). The fluid path portion may be disposable and the electronic components may be reusable. The MEMS sensors measure static and dynamic pressure of gases and liquids, such as the medicament in a closed-system (when the components are connected).

The system may also, or alternatively, utilize remote detection of fluid coupling features. This embodiment detects a pressure signal change resulting from the fluid couple disconnect and/or other events. For example, the system may utilize a closed valve or resisting membrane: such a system could employ a valve or in-tact hydrophobic membrane which would resist fluid flow during delivery if the fluid couple is disconnected. An increase in pressure, even if transient, would provide a detectable signature of a disconnect event. As another example, the system may utilize an open valve or membrane: with a portion of the infusion set disconnected, fluid resistance would drop. A decrease in pressure, even if transient, would provide a detectable signature of a disconnect event. As a more specific example, in either of these two examples, the system may be designed to include a step in which the patient confirms the detected disconnect event to a) mitigate false positives, and b) reconnect if the disconnection was not intentional.

### DETAILED DESCRIPTION OF THE FIGURES

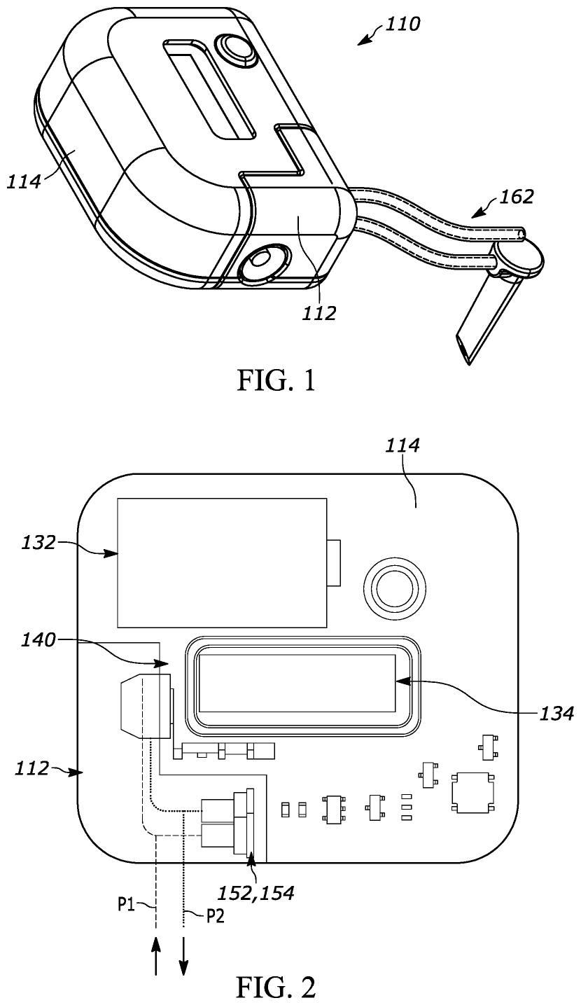

Turning to the figures, FIGS. 1 and 2 shows a drug delivery device 110 such as a pump having, generally, a disposable pump head 112, a reusable housing 114, a disposable fluid flowpath 162, a battery 132, a drive component such as a motor 140, a controller and display 134, and a pair of pressure sensors (e.g., inlet pressure transducer 152 and outlet pressure transducer 154) all contained within reusable housing assembly 114. As is further illustrated in FIG. 2, a medicament from a drug product container (e.g., an IV bag) is able to travel into the pump head 112 along the path P1 indicated with a broken line, and out of the pump head 112 along the path P2 indicated with a dotted line. In other words, the pump is able to urge the medicament through the pump head 112. The pump shown in FIG. 2 is a peristaltic pump but other suitable configurations may be used, such as a positive displacement pump. The pump head 112 shown in FIGS. 1 and 2 composed of a ring pump that utilizes a generally circular-shaped loop of tubing to create peristaltic forces. As a more specific example, the pump head has a component that pinches or otherwise occludes the ring-shaped tube section in a circular motion to urge fluid through the tube.

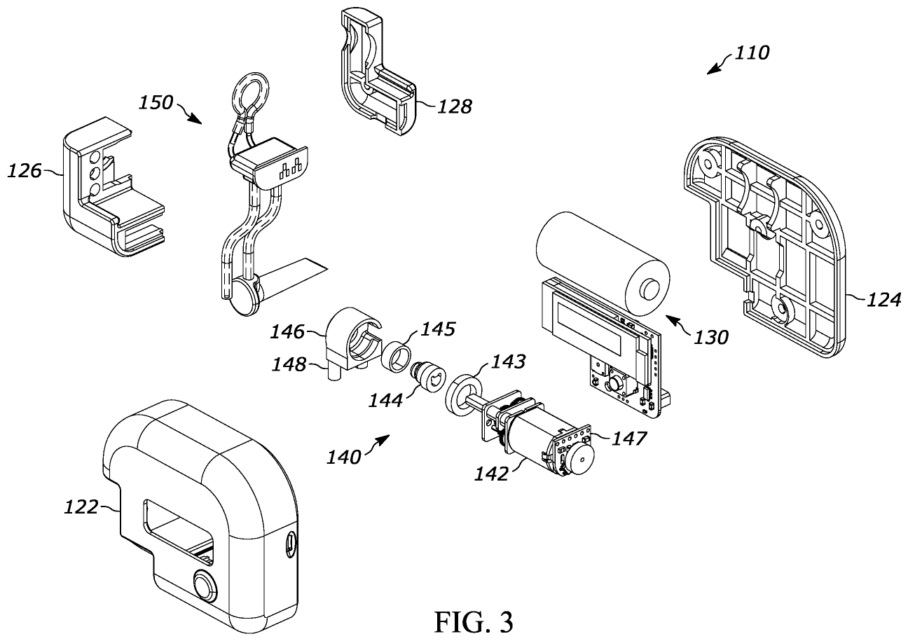

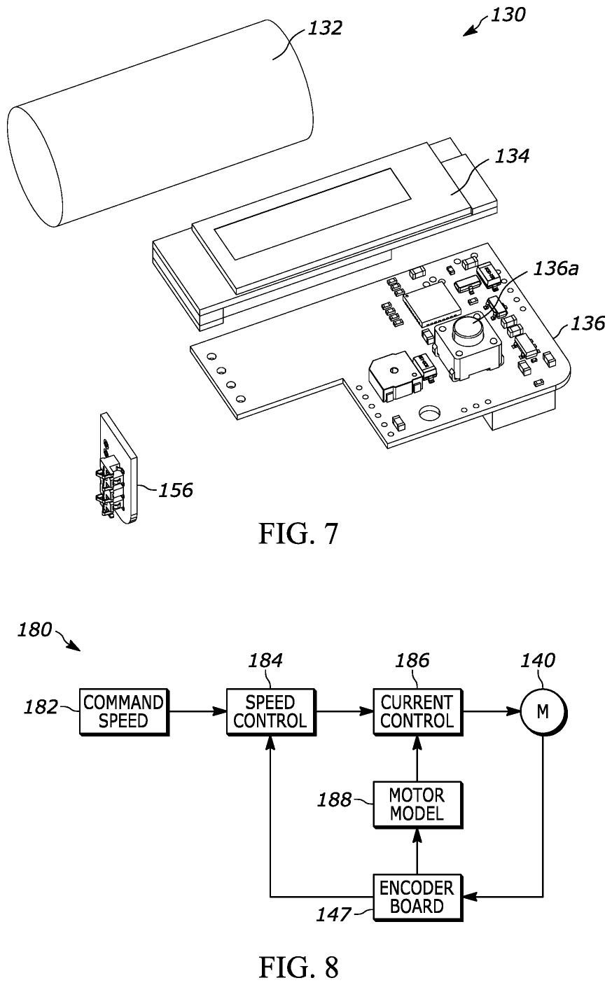

FIG. 3 shows an exploded view of the pump 110, including sub components of the housing 114, such as a controller front case 122, a controller rear case 124, a pump head front case 126, and a pump head rear case 128. These four components generally fit together to form at least the majority of the housing 114. These four components may be made of a generally rigid and lightweight material, such as plastic, a composite, or any other suitable material. The front/rear paired components (122, 124 on one hand, and 126, 128 on the other) may fit together via fasteners, snap-fit connections, an adhesive, or any other suitable coupling components/methods. A PCA and battery assembly 130 is at least partially contained within the housing 114, with a display screen 134 (FIG. 7) defining a portion of the housing 114.

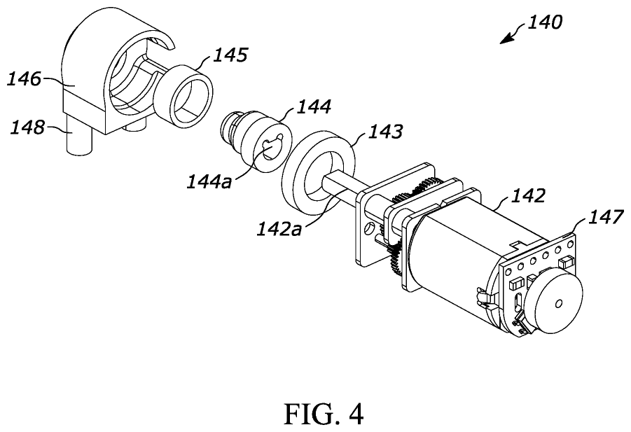

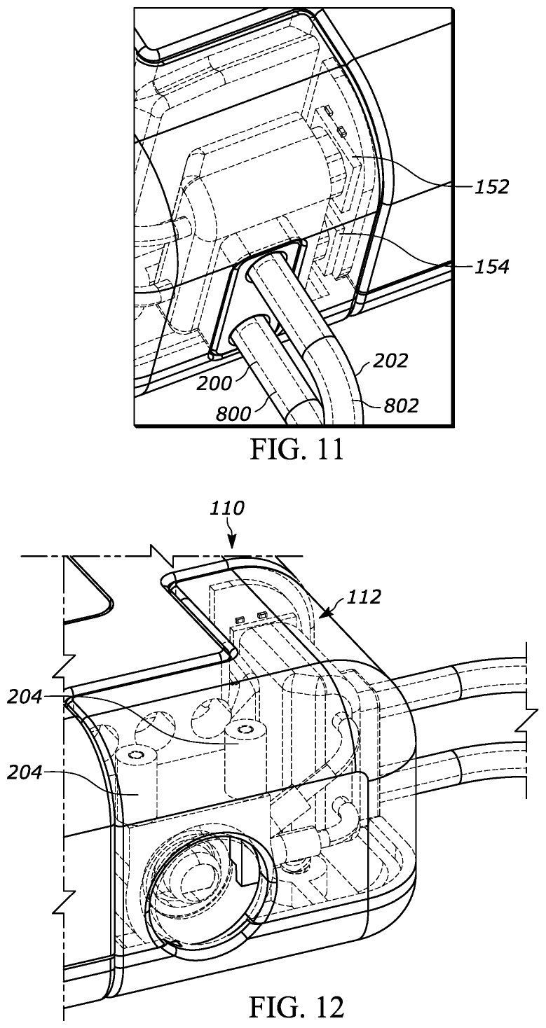

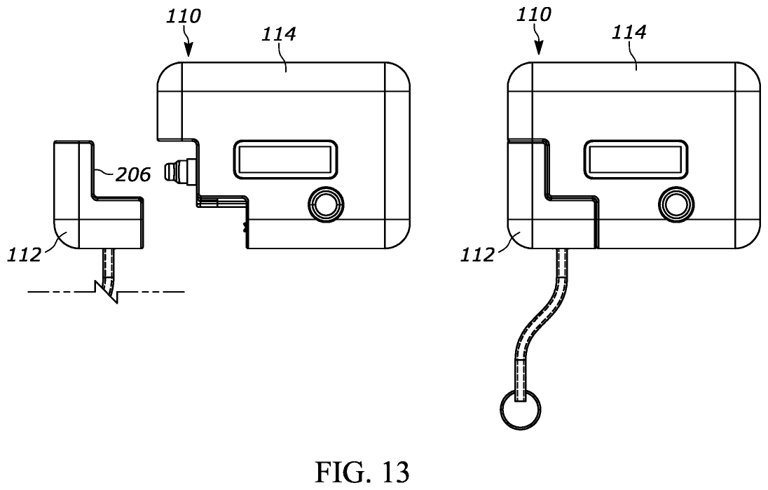

FIG. 3 further shows an exploded view of the drive assembly 140 (e.g., the motor assembly) and a tube set and pressure sensors 150. FIGS. 3 and 4 each show the exploded view of the drive assembly 140, which generally includes: a motor 142 for providing rotational drive, a retainer ring 143 for retaining other components in the housing (namely the tubes, as discussed more below), an eccentric hub 144 that utilizes a cam feature to generate peristalsis, a sleeve bearing 145 that provides a barrier between the eccentric rotor and the tubing, a pump race 146 for housing the circular-shaped tube section discussed above, an encoder board 147 for measuring the actual speed of the motor for increased accuracy and precision, and generally pliant/flexible isolation mounts 148 that prevent part misalignment, reduce drive torque/power, and provide compliance for head installation (discussed more in detail with respect to FIG. 11). As is shown in FIGS. 3 and 4, the eccentric hub 144 includes a key portion 144a that receives a correspondingly shaped drive shaft 142a. Additionally, as shown in FIGS. 13-15, the eccentric hub 144, the drive shaft 142a, the motor 142, and the encoder board 147 are located within the durable portion of the pump 112, whereas the retainer ring 143, the sleeve bearing 145, and the pump race 146 are all located within the removable pump head 112. When the pump head 112 is coupled with the durable portion 114 of the pump 110, the eccentric hub 144 lines up with and is received within the retainer ring 143. During operation, as the drive shaft 142a of the motor rotates, the eccentric hub 144 rotates about an axis that is off-set from the drive shaft axis, thereby applying an annular, outward force onto the circular-shaped tube section positioned within the pump race 146. More specifically, the retainer ring 143 fits around the circumference of the eccentric hub 144. As the eccentric hub 144 rotates, it may cause the retainer ring 143 and/or the sleeve bearing 145 to press on a relatively discrete portion of the circular-shaped tube section, thereby pinching and/or occluding that section of the tube. As the eccentric hub 144 (and the sleeve bearing) rotate further, the portion of the outer surface of the retainer ring 143 and/or the sleeve bearing 145 that is pinching the tube “rolls” around the inside of the pump race 146 and urges fluid in the tube to travel away from the pump head 112.

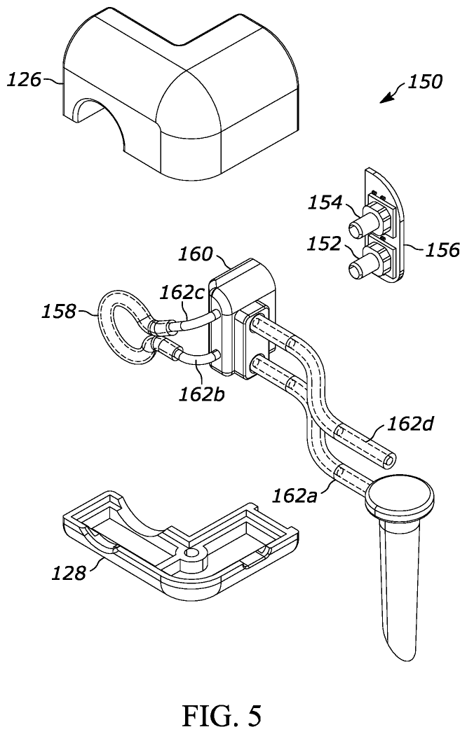

FIG. 5 shows the tube set and pressure sensors 150 in more detail, namely an exploded and enlarged view. FIG. 5 illustrates two sensors, namely inlet pressure transducer 152 and outlet pressure transducer 154, which measure fluid pressure in inlet and outlet portions of the flowpath 162. The respective transducers 152, 154 shown in the figures make contact with the flow in the manifold 160 of the pump head 112. As a more specific example, the transducers 152, 154 are electrically connected to the pump controller via sprung connector contacts and they directly measure the pressure in the flow at the inlet and outlet location. As an even more specific example, each transducer 152, 154 is electrically connected to a pressure transducer board 156 that is electrically connected to other electronic controls such as the motherboard (discussed below). For example, the transducers 152, 154 shown in the figures are each mounted on the pressure transducer board 156.

Each transducer 152, 154 shown in the figures may include a diaphragm, made from the same material as the tubing, placed inline on both the inlet and outlet tubes (162a, 162b). These diaphragms are located in the pump head 112 and make contact with a portion of the pump controller (e.g., the pressure transducer board) when the pump head assembly is installed via the pressure transducer board 156. At the point of diaphragm contact, load cells in the pump controller monitor variation in force exerted by the diaphragm which correlates to pressure changes in the flow. In this manner, the flow rate can be monitored at the inlet and outlet of the pump head 112 which provides the pressure sensor benefits discussed herein without introducing any new materials into drug contact. One or both of the transducers 152, 154 may be workingly connected to the controller such that the controller is able to detect a disconnection of the fluid path based on changes detected by/values measured by the transducers.

Other or alternative types of pressure sensors may be utilized, such as non-contact pressure sensors design to provide the benefits of pressure sensors but without the risk of material non-compatibility.

FIG. 5 also shows an example of the fluid flowpath 162 in more detail. For example, the fluid flowpath 162 may include an external tubing inlet side portion 162a, an internal tubing inlet side portion 162b, an internal tubing outlet side portion 162c, and an external tubing outlet side portion 162d. The various portions of tubing 162a-d may be integrally formed (i.e. a single piece of tubing), or they may be made of two or more sections of tubing that are fluidly connected with each other. The external tubing portions 162a, 162d shown in the figures are each formed of the same type and sized tubing as each other and potentially the same type and sized tubing as IV lines. The internal tubing portions 162b, 162c shown in the figures are each formed of a smaller diameter tube to facilitate pressure measurement. The flowpath 162 also include a ring tubing 158, i.e., the generally circular portion of tubing discussed above that is housed within the pump race 146. In one embodiment, the ring tubing 158 defines the boundary between the inlet fluid flowpath and the outlet fluid flowpath. As discussed above, the pump head 112 components shown in FIG. 5 are supported by the pump head front and rear case 126, 128 and the pump head 112 is removably coupled with the remainder pump structure. The pump head 112 may be disposable and the remainder pump structure may be reusable (e.g. “durable”).

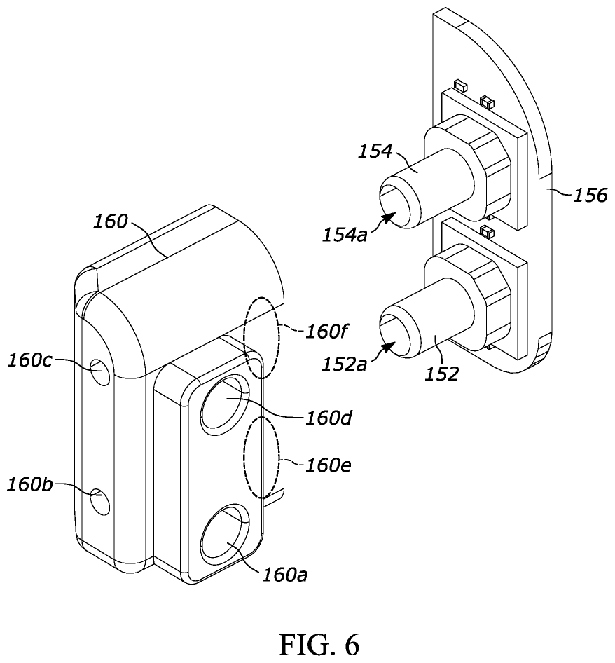

FIG. 6 shows an enlarged view of the tubing manifold 160 and the pressure sensors (152, 154, 156). The transducers 152, 154 are inserted within transducer ports (shown with dotted lines 160e and 160f) on the side of the manifold 160 so as to measure fluid pressure within the tubes that extend through the manifold 160. For example, the manifold includes manifold port external inlet 160a for receiving the external tubing inlet side portion 162a, manifold port internal inlet 160b for receiving the internal tubing inlet side portion 162b, manifold port internal outlet 160c for receiving the internal tubing outlet side portion 162c, and manifold port external outlet 160d for receiving the external tubing outlet side portion 162d. The transducer ports 160e, 160f are in-line with the other ports and are sized to receive the transducers 152, 154 and may be in fluid communication with the other ports. For example, ports 160a, 160b, and 160e are in fluid communication with each other such that the inlet fluid flow travels through tubing 162a, into contact with the diaphragm (the location of which is indicated by arrow 152a) of transducer 152, into tubing 162b, through the ring tubing 158, through tubing 162c, into contact with the diaphragm (the location of which is indicated by arrow 154a) of transducer 154, and into tubing 162d.

FIG. 7 shows the PCA and battery assembly 130, including a battery 132, an OLD display 134 (or alternatively LED, OLED, or LCD), a motherboard 136, and the pressure transducer board 156. As discussed above, the pressure transducer board 156 is electrically coupled with the motherboard 136 so the respective components can exchange inputs and outputs. Exemplary battery types and specifications are shown in FIGS. 28-29. The OLD display 134 may display user and/or pump information. The motherboard 136 may include a pushbutton 136a for operating the device, such as triggering a start or stop cycle. The motherboard 136 may host different functions and/or controls such as motor control; pulse with modulation (PWM) control; Proportional, Integral, Derivative (PID) control to stabilize the pump, sound control, user input control, and encoder board/speed control.

FIG. 8 shows a flowchart of one exemplary operation of a controller 180 of the pump 110 that has improved accuracy and/or precision. The controller 180 may include the motherboard 136 and/or other components. For example, the controller shown in FIG. 8 includes an encoder-fed, closed loop system. As a more specific example, the controller shown in FIG. 8 includes an encoder board 147 for determining measured drive speed and a motor model 188 for determining a calculated drive speed. In this example, the controller is configured to adjust at least one parameter of the drive component based on the measured drive speed and the calculated drive speed. For example, during operation, the motherboard and/or a user input may dictate a desired speed for the motor 140, i.e., a “command speed” 182. The command speed 182 is then inputted into a “speed control” 184 component that may be coupled to or integrally formed in the motherboard 136. The speed control then sends input to the “current control” 186 which in turn sends a certain amount of current to the motor 140. An encoder board 147 is mounted to a portion of the motor 140 or adjacent to a portion of the motor 140 to measure the speed of a rotary portion of the motor. This measured speed information (e.g. measured drive speed) is then inputted back to the speed control 184. At the same time, the encoder board inputs the measured speed information to a motor model 188, which calculates a calculated (or predicted) drive speed based on operating conditions. The speed control 184 and the current control 186 each are able to receive and process these inputs and potentially vary their operation based on the same. For example, if the calculated speed from the encoder board 147 differs from the command speed 182, then the respective components may be able to adjust the current level sent to the motor 140 to more accurately and precisely operate at (or near) the command speed. The controller 180 may also receive inputs from the pressure sensors 152, 154 as part of the feedback/control system.

This feedback/control system may allow the pump 110 to operate at a high accuracy. For example the controller 180 may be configured such that the pump is able to deliver medicament at an accuracy rate of at least 95%. More specifically, the controller 180 may be configured such that the pump is able to deliver medicament at an accuracy rate of at least 97%. Even more specifically, the controller 180 may be configured such that the pump is able to deliver medicament at an accuracy rate of at least 98%. Even more specifically, the controller 180 may be configured such that the pump is able to deliver medicament at an accuracy rate of at least 99%. The controller 180 may be configured such that the pump is able to deliver medicament at one or more of these accuracy levels during delivery of a dose of the medicament having a volume of at least 200 milliliters or 250 milliliters. This feedback/control system may allow the pump 110 to operate at a high efficiency, thereby maximizing battery life, reducing device noise and vibration, reducing generated motor heat, and/or improving overall performance. The feedback/control system may allow the pump to operate at the accuracy levels discussed herein despite varying operating conditions, such as vertical height differential (positive or negative) between the pump and the drug product container. For example, the pump has been tested to maintain accuracy at +/−36 inches between the pump and the drug product container.

FIG. 9 shows an exemplary drug delivery assembly 100 (or “system”) for use with the pump 110. For example, the assembly 100 shown in FIG. 9 includes a drug product container 102 for containing a drug product 102a (or medicament), an IV input line 104a, an IV output line 104d, a pair of connectors/adaptors 108a, 108b, and the tubing portions 162a, 162d leading to and from the pump 110. As a more specific example, the connection points may include quick-connect sterile connectors with respective sub-components that selectively mate with each other while maintaining sterility or another desirable cleanliness standard. For example, the quick-connect sterile connectors may snap or twist or screw together; they may have sheathed or covered components that become unsheathed or uncovered upon connection; and/or they may have Luer Lock or modified Luer Lock configurations. As another example, the connectors may include one or more stake connectors for coupling one of the tube 162 portions with an IV bag. The distal end of the IV output line 104d may also include or be coupled with a drug delivery connector (not shown) such as a needle, a luer lock component, or another suitable component. As shown in FIG. 9, an IV spikes may pierce the port of the drug container 102 to physically connect the drug product container to the fluid path assembly 160. Additionally or alternatively, one or more of the connectors/adaptors 108a, 108b may include sensors positioned within or adjacent to respective components of the connectors/adapters 108a, 108b. The sensors may be configured to detect pressure changes and/or upper/lower range values that are indicative of a fluid path disconnection event. For example, FIG. 10 shows one such exemplary sensor, a sensor/adapter 108 such as a MEMS-based sensor incorporated into or around the tubing sets. Alternatively, any suitable sensor and/or configuration may be used.

The adaptors may be sterile quick-connect components. Example CSTD devices may include the OnGuard CSTD provided by B. Braun Medical Inc, BD PhaSeal CSTD components, Equashield CSTD, Codon CSTD, and the like. Further, non-closed system transfer devices may be used such as West Pharmaceuticals vial and bag adapters. Other examples are possible. The prefilled delivery container may include any number of delivery container adapters having different specifications (e.g., port sizes) to accommodate the use of different drug product vials.

FIG. 11 shows a more detailed view of the pressure sensors 152, 154 along the fluid path and how they may be used to provide occlusion detection, end-of-bag detection, and IV bag pressure compensation. For example, the inlet line 200 (from the IV bag) can include an inlet pressure 800 that may be used to compensate pump run time for improved dose accuracy and/or to detect an empty bag. As another example, the outlet line 202 can include an outlet pressure 802 that may be used to detect occlusions.

As shown in FIG. 12, the removable pump head 112 may be compliantly mounted with respect to the durable portion of the pump 110 via rubber isolation mounts 204, or other compliant mount components, to allow for easier installation and it to reduce motor voltage spikes due to component tolerancing.

FIG. 13 shows a view of the pump head 112 in the non-attached (left) and attached (right) configurations. The non-attached configuration may allow for manual priming of the infusion set (i.e., squeezing the IV bag until the inlet flowpath is substantially or completely filled with drug product and air has been substantially or completely evacuated therefrom). The non-attached configuration may also be desirable for storage to prevent or minimize the likelihood of tube compression damage. The pump head 112 may be slid from the non-attached position shown on the left, to the attached position shown on the right, onto the reusable housing 114 and may be secured in place via magnets 206 or other suitable coupling features.

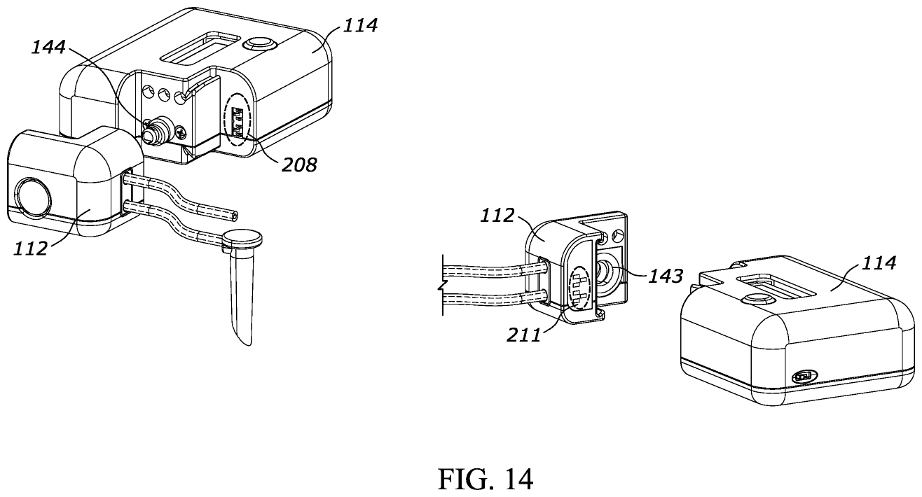

FIG. 14 shows an exemplary design for the pressure sensor interface with the controls, where the housing 114 sensor contacts 208 the cooperate with sensor board interface “pads” 211 on the head 112.

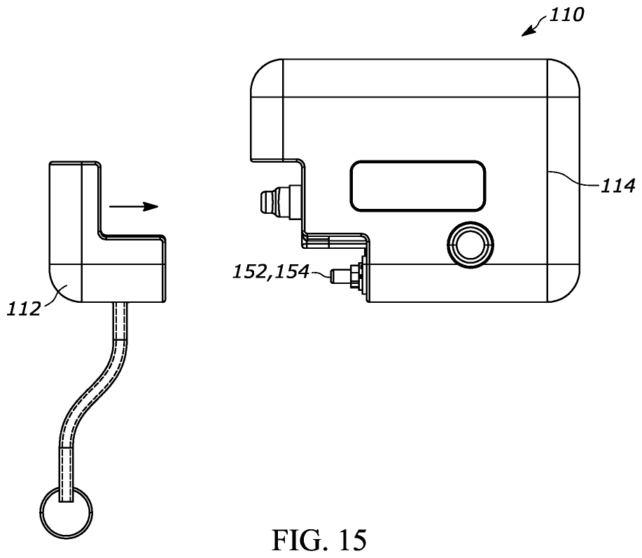

FIGS. 15 and 16 show exemplary designs, with the pressure sensors 152, 154 are located on the durable unit or reusable housing 114 rather than on the removable pump head 112.

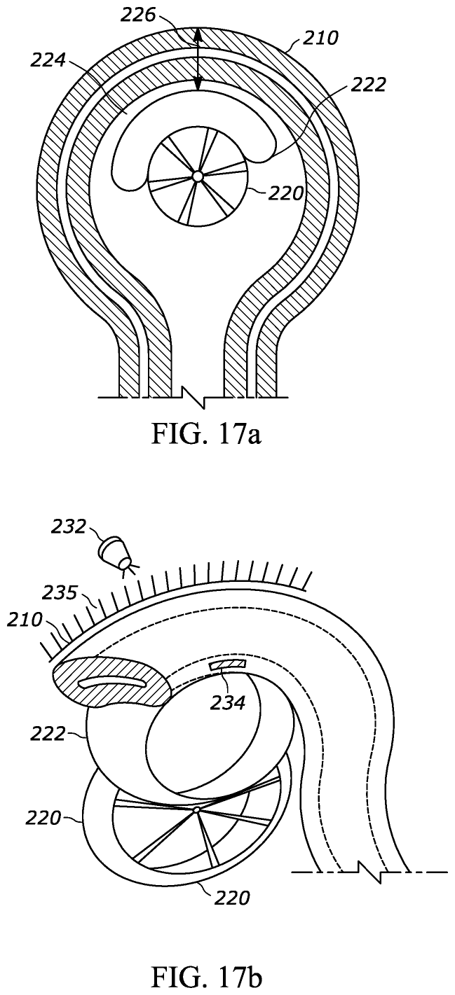

FIG. 17a shows a ring tube portion 210, a drive component 220 (e.g., a rotor element) having an eccentric component 222 (e.g. a ring spring), where the eccentric component 222 has a contact surface 224 that compresses the ring tube portion 210 by a compression distance 226. FIG. 17b shows a compression sensor workingly coupled with the eccentric component 222 and configured to measure input relating to and/or proportional to the compression distance 226. For example, FIG. 17b shows a light emitter 232 and a CMOS detector 234 that are placed on opposite sides of the ring tube portion 210 to register the amount of light transmitted. For example, the light emitter 232 is positioned in or near a pump race housing 235 and the CMOS detector 234 is placed on an opposite surface of the ring tube portion 210. However, any suitable configuration may be used. As the ring tube portion 210 is squeezed between the eccentric component 222 and the pump head, an inner diameter of the ring tube portion 210 is distorted changing the transmission and refractive indices of the ring tube portion 210. This inner diameter displacement can be related to volume of fluid being dispensed per cycle of rotation of the rotor element 220 as shown in FIGS. 17a-b. The controller then could adjust the speed of rotation of the rotor element 220 in real-time to achieve the desired flow rate.

FIG. 18 shows a ring tube portion 310, a drive component having an eccentric component (e.g. a ring spring), where the ring spring has a contact surface that compresses the ring tube portion 310 by a compression distance 326. In this configuration, an active ring utilizing actuator(s) 331 such as PZT element(s) or any other form known to the art may be used to modulate the pressure applied on the tube 310 to achieve full collapse during rotation of the pump-head rotor 333. FIG. 18 shows a compression sensor workingly coupled with the eccentric component and configured to measure input relating to and/or proportional to the compression distance. For example, FIG. 18 shows electrode elements 328, 330 that are embedded into the small segment of the tubing 310 in the inner diameter of the tube 310. When the tube 331 is fully squeezed so that inner diameter is fully collapsed and the two electrodes 328, 330 touch, a short circuit can be established to close a circuit as the trigger to indicate sufficient pressure applied to the tube to achieve the desired flowrate. The controller then could adjust the speed of rotation of the rotor in real-time to achieve the desired flow rate.

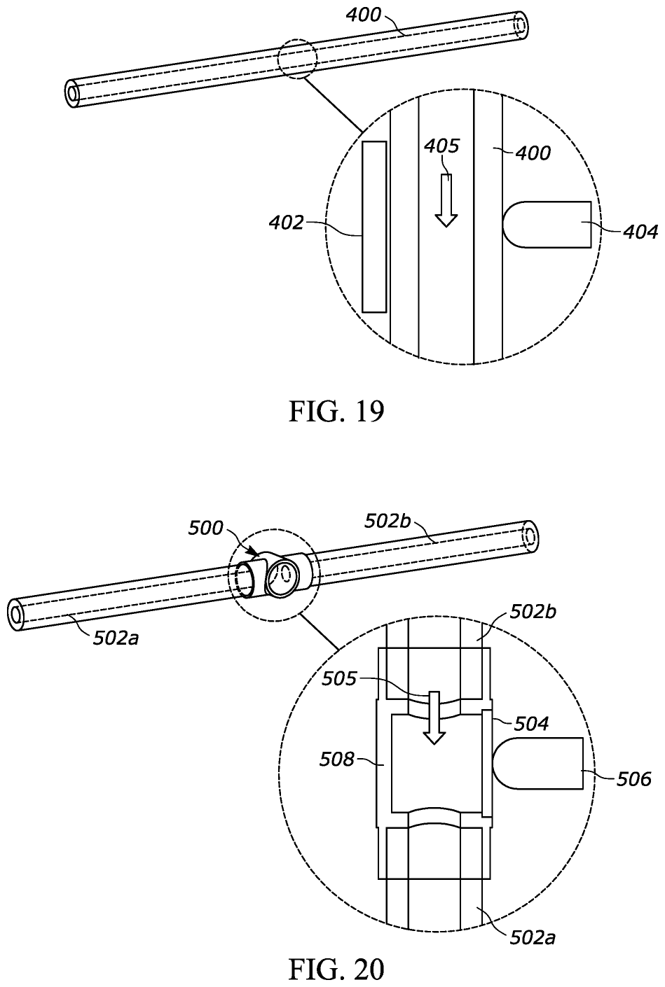

FIG. 19 shows an alternative design for the type of pressure sensor utilized, namely one that can be utilized with a conventional IV tube 400 containing a flow 405 of medicament, for example, therethrough. The pressure sensor in FIG. 19 includes a tubing support 402 and a sensor contact 404 positioned in-line with the tubing support 402 such that as pressure increases or decreases and the IV tube 400 expands or contracts in small increments, the sensor contact 404 is able to measure the same and detect flow pressure.

FIG. 20 shows another alternative design for the type of pressure sensor utilized, namely one that includes a connector 500 between two sections of conventional IV tubing 5021, 502b containing a flow 505 of medicament, for example, therethrough. The connector 500 has a rigid frame 508 that has a flowpath therethrough, a flexible diaphragm 504 fluidly connected with the flowpath such that the diaphragm 504 moves inward or outward based on the flow pressure therethrough, and a sensor contact 506 for measuring the position of the diaphragm 504.

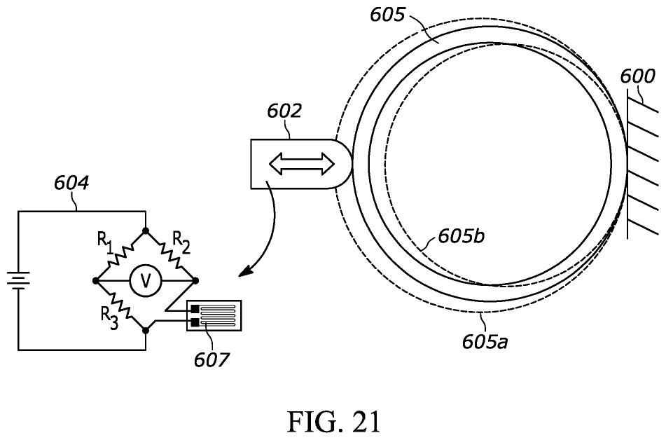

FIG. 21 shows the schematic design for the type of non-contact pressure sensor utilized, namely one that a portion of the flowpath (IV tubing or diaphragm 605) described above that is able to expand under increased pressure to an expanded cross-section 605a or contract under decreased pressure to a reduced cross-section 605b, a support 600 as positional reference, a sensor actuator 602 to detect motion (expansion or contraction) with respect to the reference support 600, and sensor electronics 604 including a strain gauge 607, for example, for responding to the sensor actuator 602, whereby the electronics communicate with the controller.

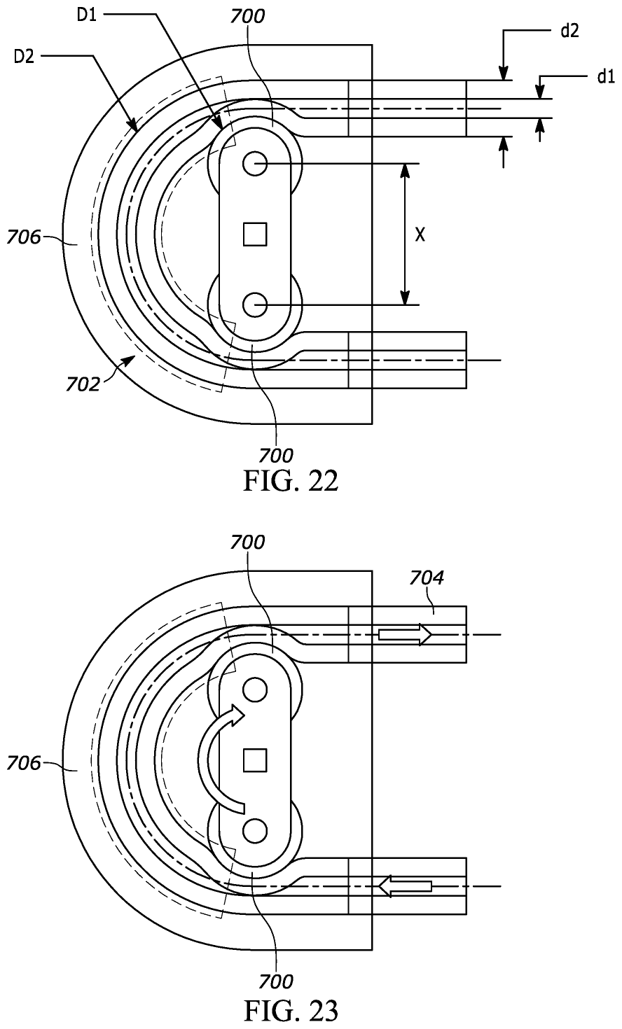

FIGS. 22 and 23 show an alternative design for the type of pump utilized, namely a roller-based peristaltic pump having two (or more) rollers 700 that cooperate to define a trapped volume of fluid 702 and then urge the trapped volume through the tube 704. Each roller 700 has a roller diameter D1 and a tube support 706, supporting the tub 704, includes a diameter D2 larger than D1. The tube 704 includes an inner diameter and an outer diameter d2. In the depicted version, the rollers 700 are spaced by a roller spacing X.

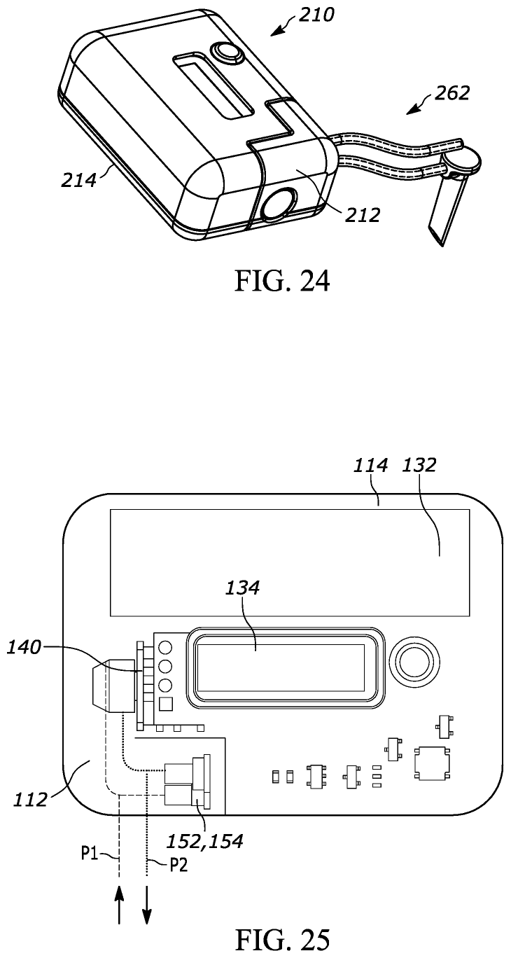

FIGS. 24-25 show an alternative design for the pump 210, having similar components as shown in FIG. 1, but with a larger capacity and operational life. For example, the pump 110 shown in FIG. 1 may be utilized for weekly use (i.e., up to one week) whereas the pump 210 shown in FIGS. 24 and 25 may be utilized for monthly use (i.e., up to one month).

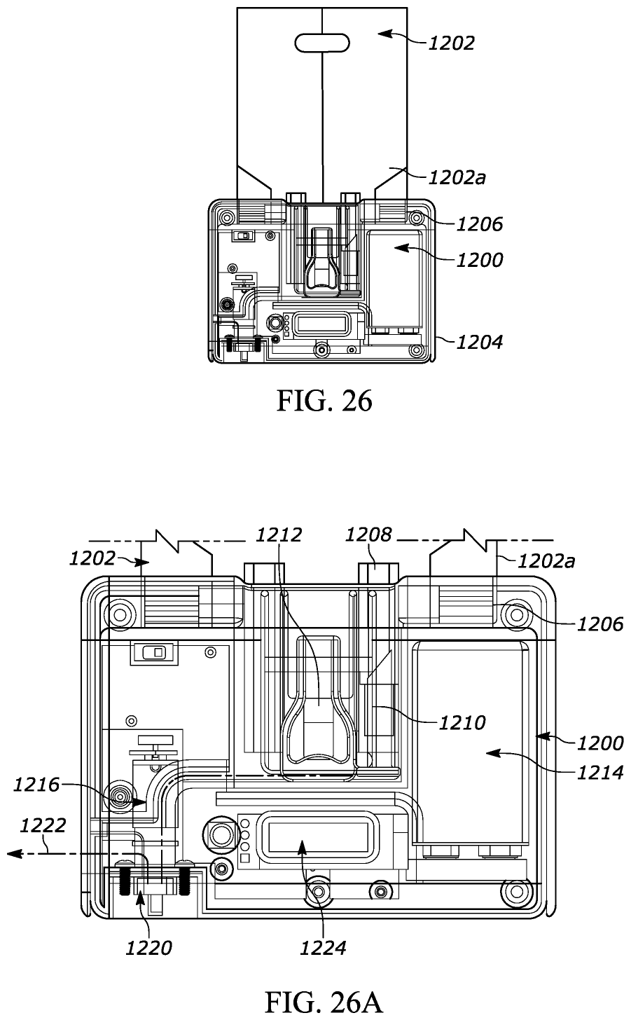

FIG. 26 shows another alternative design, where the pump has a clip-in or other physical connection between the pump and the IV bag.

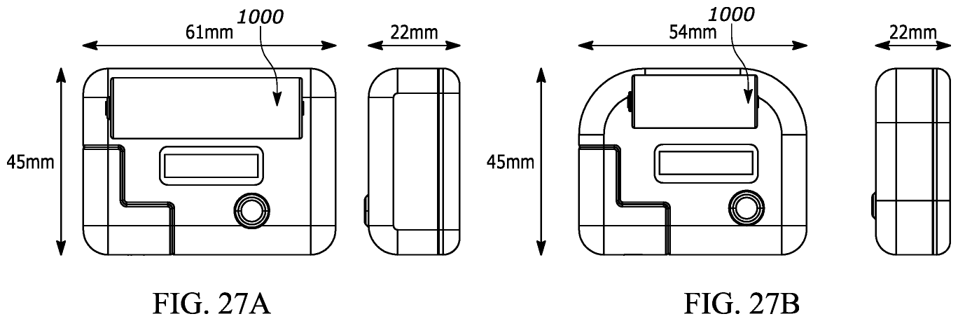

Based on the foregoing, FIGS. 27A and 27B illustrates two exemplary drug delivery devices in accordance with various embodiments of the disclosure incorporating different form factors, geometries, dimensions, and battery sizes. The embodiment in FIG. 27A, as shown, is generally rectangular in profiles and is approximately 45 mm high, 61 mm wide, and 22 mm thick, and includes an AA Battery 1000 (such as AA LS-14500-BA) suitable for monthly delivery. In contrast, the embodiment in FIG. 27B, as shown, is also generally rectangular in profile but with an arc-shaped top. This embodiment is 45 mm high, 54 mm wide, and 22 mm thick, and includes a ⅔ AA Battery 1000 (such as ⅔AA ER14335) suitable for weekly delivery. The following tables (Table 1 and Table 2) illustrate various exemplary technical specifications for these exemplary batteries for drug delivery devices in accordance with various embodiments including, for example, an embodiment with a monthly battery (Table 1) compared to an embodiment with a weekly battery (Table 2).

TABLE 1
Monthly Battery Technical Specifications
Monthly Battery: AA LS-14500-BA

| Chemistry Type | Lithium Thionyl Chloride |
|---|---|
| Rechargeable | No |
| Voltage (V) | 3.6 |
| Capacity (Ah) | 2.6 |
| Continuous Discharge (mA) | 100 |
| Energy (J) | 33696 |
| Power Capacity (Wh) | 9.36 |
| Diameter (mm) | 14.5 |
| Height (mm) | 50.5 |
| Volume (mm\hat{ }3) | 8339.1 |

TABLE 2
Weekly Battery Technical Specifications
Weekly Battery: 2/3AA ER14335

| Chemistry Type | Lithium Thionyl Chloride |
|---|---|
| Rechargeable | No |
| Voltage (V) | 3.6 |
| Capacity (Ah) | 1.65 |
| Continuous Discharge (mA) | 50 |
| Energy (J) | 21384 |
| Power Capacity (Wh) | 5.94 |
| Diameter (mm) | 14.5 |
| Height (mm) | 33.5 |
| Volume (mm\hat{ }3) | 5531.9 |

Table 3 (below) further demonstrates the performance characteristics of exemplary drug delivery devices incorporating batteries from Tables 1 and 2. The data demonstrates that power consumption varies. In some embodiments, power consumption is seen as low as 225 mW while pumping fluid. In other embodiments, power consumption is seen as high as 540 mW while pumping fluid. 248 mW to 360 mW was the median range of power consumption.

TABLE 3
Device Performance

| Pump Characteristics | Weekly | Monthly |
|---|---|---|
| Drug Volume (mL) | 100 | 400 |
| Delivery (days) | 7 | 28 |
| Required Aliquot Rate (mL/hr) | 0.60 | 0.60 |
| Pump Delivery 100% (mL/min) | 0.59 | 0.59 |
| Duty Cycle | 1.00 | 1.00 |
| Pump on Time (min/hr) | 1.01 | 1.01 |
| Pump Standby Time (hr) | 167.60 | 671.60 |
| Pump Pumping Time (hr) | 7.06 | 28.25 |
| Battery PN | ER14335 | LS-14500 |
| Nominal Voltage | 3.6 | 3.6 |
| Stated Battery Capacity (mAh) | 1200 | 2600 |
| Cutoff Voltage | 2 | 2 |
| Measured Battery Voltage (V) @ 60 mA | 2.89 | 3.22 |
| Capacity @ 60 mA (mAh) | 788 | 1131 |
| Usable Battery Capacity (mWh) | 2837 | 4072 |
| Boost Voltage | 4.5 | 4.5 |
| Boost Efficiency | 0.8 | 0.8 |
| Usable Booted Battery Capacity (mWh) | 2269 | 3257 |
| System Pumping Power (mW) | 247.5 | 247.5 |
| System Idle Power (mW) | 1.8 | 1.8 |
| Estimated Possible Pumping Time (hrs) | 9.17 | 13.16 |
| Estimated Capacity Used in 1 Hr (mWh) | 5.93 | 5.93 |
| Estimated Battery Life (hrs) | 382.62 | 549.16 |
| Estimated Battery Life (Days) | 15.94 | 22.88 |

The various components, devices, embodiments, and systems described may be advantageous over known components, devices, and systems for a number of reasons. For example, the pump designs and/or embodiments disclosed herein have a reduced, size, weight, and overall footprint compared to known pump designs. This advantage may offer dramatic quality of life and/or convenience for patients using the pump designs. As another example, the pump designs and/or embodiments disclosed herein may have an improved dose accuracy. As yet another example, the pump designs and/or embodiments disclosed herein may have a reduced complexity of the device and overall system. As yet another example, the pump designs and/or embodiments disclosed herein may have a reduced pump noise. As yet another example, the pump designs and/or embodiments disclosed herein may have a reduced cost of the device and the overall system. As yet another example, the pump designs and/or embodiments disclosed herein may have increased reliability of the device and overall system. As yet another example, the pump designs and/or embodiments disclosed herein may have an increased product life of the device and overall system.

It may be desirable to utilize components that allow for fast/easy/sterile connections/disconnections. The fluid flowpath may be defined by a sterile single-use tubing system and valve system. The system may be used to provide intravenous, subcutaneous, intra-arterial, intramuscular, and/or epidural delivery approaches. By using the system, patient anxiety and or confusion may be reduced due to reduced preparation complexity and wait times caused by the drug preparation process.

In some examples, the system may be utilized with medicament in the form of a half-life extended bispecific T cell engager (BiTE®). For example, the active pharmaceutical ingredient (“API”) may be between approximately 2 mcg and approximately 100 mcg, depending on the BiTE® and container size, which, may be in a powdered form (i.e., lyophilized) requiring reconstitution. In other examples, the drug product may be in liquid form and may not require reconstitution. Nonetheless, the system includes an accurate quantity of drug product, and thus does not require the need to add additional quantities thereto in a sterile environment. In some examples, the API may be in the form of a half-life extended (“HLE”) BiTE® and/or an IV-admin monoclonal antibody (“mAbs) as desired. These HLE BiTE®s include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BiTE® that is to be administered in a professional healthcare environment.

The medicament may also include other components such as an IVSS, saline solution, and/or a diluent. The IVSS may include polysorbate. In some examples, the IVSS formulation may include approximately 1.25 M lysine monohydrocholoride, 25 mM citric acid monohydrate, 0.1% (w/v) polysorbate 80, and has a pH of approximately 7.0. In other examples, the IVSS 54 may include similar formulations, but also have a minimum of approximately 0.9% NaCl and approximately 0.001 to approximately 0.1% (w/v) polysorbate 80. It is appreciated that different BiTE®s require different final percentages of IVSS 54 in the delivery container. This percentage may vary between approximately 0.5% to approximately 12% of the final volume in the delivery container. Further, citrate may increase the risk of glass delamination if filled in glass vials. In the event that citrate is necessary for drug product stabilization (determined on a per-product basis), the delivery containers may be constructed from CZ or other plastic compositions. Other examples of ingredients for suitable IVSSs are possible. Suitable IVSS concentrations protect against protein-plastic interactions and/or surface adsorption, and more specifically, in the lower end of the concentration range where even minor losses may potentially change the effective dose. The below table illustrates example component concentrations for varying IVSS concentrations:

TABLE 4
Component Concentrations with Varying IVSS
Concentrations (top column units are (V/v) % of IVSS

| IVSS | | | | | | | | |
|---|---|---|---|---|---|---|---|---|
| COMPONENTS | 0.5 | 1.0 | 2.0 | 4.0 | 6.0 | 8.0 | 10.0 | 12.0 |
| Lysine monohydro- | 0.00625 | 0.0125 | 0.025 | 0.05 | 0.075 | 0.1 | 0.125 | 0.15 |
| chloride (M) | | | | | | | | |
| Citrate | 0.000125 | 0.00025 | 0.0005 | 0.001 | 0.0015 | 0.002 | 0.0025 | 0.003 |
| Monohydrate (M) | | | | | | | | |
| Polysorbate 80 | 0.0005 | 0.001 | 0.002 | 0.004 | 0.006 | 0.008 | 0.01 | 0.012 |
| (% w/V) | | | | | | | | |

By providing the components in containers that are selectively connectable, it may be no longer necessary to prepare a needle and syringe assembly to inject one component into another container, to ensure that this prepared needle and syringe assembly is sterilized, and/or to ensure a correct volume or amounts of components are added together.

In some embodiments, the drug delivery system may have an integrated reconstitution subsystem onboard to dilute a lyophilized drug into a liquid form. In certain such embodiments, a diluent reservoir may be included for storing a diluent solution and a lyophilized reservoir may be included storing a lyophilized compound separate from the diluent solution. Furthermore, a fluid drive mechanism may be included for mixing the diluent solution in the diluent reservoir with the lyophilized compound in the lyophilized reservoir. In some embodiments, the fluid drive mechanism may transfer the diluent solution from the diluent reservoir into the lyophilized reservoir and/or provide any circulation and/or agitation needed to achieve full reconstitution. In some embodiments, an additional final reconstituted drug reservoir may be included and serve as a delivery reservoir from which the reconstituted drug is discharged into the patient; whereas, in other embodiments, the lyophilized reservoir may serve as the delivery reservoir. While the reconstitution subsystem may be physically integrated into the drug delivery system in certain embodiments, in other embodiments the reconstitution subsystem may constitute a separate unit which is in fluid communication with the drug delivery system. Having a separate unit may simplify the reconstitution process for healthcare providers in certain cases.

The drug product container may be in the form of an IV bag, a vial, a prefilled syringe, or similar container that includes a reconstitution container body defining an inner volume. The inner volume may be sterile. In some approaches, the reconstitution container adapter may also be a CSTD (or, in examples where the prefilled reconstitution container is in the form of a syringe, the container adapter may be a needle) that mates, engages, and/or couples to the vial adapter. Additionally or alternatively, the drug product can be bulk lyophilized and filled into a cartridge or container that is typically used to administer with an IV pump. If needed the dehydrated forms of IVSS, NaCl, and any other components needed for the final administered solution can be bulk lyo'ed and filled into the cassette for long term storage.

As previously noted, in some examples, the prefilled drug product container may be in the form of a prefilled syringe that contains the drug product. In these examples the drug product may be in the form of a liquid BiTE® formulation used in conjunction with a monoclonal antibody (mAb), In these examples, the drug product may be directly added to the delivery container without the use of a vial adapter system (such as the above-mentioned CSTDs) where more traditional needle-syringe injection/delivery into the container is preferred, which may advantageously simplify and/or improve supply chain and manufacturing control, and may further allow for more compact commercial packaging that takes up less space in storage systems at healthcare facilities. In these examples, the prefilled drug product vial may or may not need to be reconstituted prior to transferring the drug product to the delivery container.

The system may be distributed and/or sold as a common kit packaging, but other suitable distribution/packaging is suitable. The drug product may be in the form of a half-life extended bispecific T cell engager (BiTE®), but other drug products are suitable. The diluent include water for injection (“WFI”), but other diluents may be suitable. The containers may be pliable bags, such as IV bags, but other containers may be suitable. In some examples, one or more of the containers is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage.

In some examples, the prefilled delivery container is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container. Example suitable prefilled delivery containers are described in U.S. Appln. No. 62/804,447, filed on Feb. 12, 2019 and U.S. Appln. No. 62/877,286 filed on Jul. 22, 2019, the contents of each of which are incorporated by reference in their entirety.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RAN KL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“ID-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-?4ß37 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNF? monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-?4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2R? mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNF? mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-?5?1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFN? mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG? mAb (MDX-1307); anti-mesothelin dsFv-PE38 conjugate (CAT-5001); anti-PD1mAb (MDX-1106 (ONO-4538)); anti-PDGFR? antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TI MP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF? monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™ or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device for delivering a medicament, comprising:
a housing;
a pump coupled with the housing;
a drive component for driving the pump;
an inlet fluid path configured to deliver medicament to the pump;
an outlet fluid path configured to receive medicament from the pump;
an inlet pressure sensor positioned along the inlet fluid path and configured to measure inlet fluid pressure;
an outlet pressure sensor positioned along the outlet fluid path and configured to measure outlet fluid pressure; and
a controller workingly coupled with the inlet pressure sensor, the outlet pressure sensor, and the drive component, wherein the controller is configured to adjust at least one parameter of the drive component based on input information received from the inlet pressure sensor and/or the outlet pressure sensor.

2. The drug delivery device as in claim 1, wherein the controller is configured to detect (a) an occlusion event based on the input information received from the inlet pressure sensor and/or the outlet pressure sensor, and/or (b) a low medicament event based on the input information received from the inlet pressure sensor and/or the outlet pressure sensor.

3. (canceled)

4. The drug delivery device as in claim 1, further comprising a pump manifold supporting at least a portion of the inlet fluid path and at least a portion of the outlet fluid path;
wherein the inlet pressure sensor and/or the outlet pressure sensor are supported by the pump manifold.

5. The drug delivery device as in claim 1, wherein the inlet pressure sensor and/or the outlet pressure sensor includes a transducer.

6. The drug delivery device as in claim 1, wherein the medicament is in the form of a bispecific T cell engager (BiTE®), wherein the BiTE® is optionally a half-life extended (HLE) BiTE®.

7. (canceled)

8. The drug delivery device as in claim 1, wherein the at least one parameter of the drive component is the speed of the drive component.

9. The drug delivery device of claim 1, further comprising:
a medicament container containing a medicament;
wherein the inlet fluid path is further configured to receive the medicament from the medicament container;
wherein the outlet fluid path is further configured to deliver the medicament to a patient.

10. The drug delivery device as in claim 9, wherein the drug delivery device defines a boundary between the inlet fluid path and the outlet fluid path.

11-12. (canceled)

13. The drug delivery device as in claim 9, further comprising at least one adaptor for fluidly connecting (a) at least two sections of the inlet fluid path with each other and/or (b) at least two sections of the outlet fluid path with each other.

14. (canceled)

15. The drug delivery device as in claim 9, further comprising a delivery member configured to fluidly connect with an IV connector.

16. The drug delivery device as in claim 9, wherein the inlet pressure sensor and/or the outlet pressure sensor includes a transducer.

17. The drug delivery device as in claim 9, wherein the at least one parameter of the drive component is the speed of the drive component.

18. A drug delivery device for delivering a medicament, comprising:
a housing;
a pump coupled with the housing;
a drive component for driving the pump;
a fluid path workingly coupled with the pump such that the pump is configured to urge the medicament along the fluid path;
at least one sensor configured to measure a flow parameter of the medicament along the fluid path;
a controller workingly coupled with the at least one sensor and the drive component, wherein the controller is configured to adjust at least one parameter of the drive component based on input information received from the at least one sensor; and
wherein the controller is configured to deliver the medicament at an accuracy rate of at least 95%.

19. A drug delivery device as in claim 18, wherein the controller includes an encoder-fed, closed loop system.

20. A drug delivery device as in claim 19, further comprising an encoder board for determining measured drive speed and a motor model for determining a calculated drive speed;
wherein the controller receives input relating to the measured drive speed and the calculated drive speed; and
wherein the controller is configured to adjust the at least one parameter of the drive component based on the measured drive speed and the calculated drive speed.

21. A drug delivery device as in claim 18, wherein the controller is configured to:
(a) deliver the medicament at an accuracy rate of at least 97%,
(b) deliver the medicament at an accuracy rate of at least 98%,
(c) deliver the medicament at an accuracy rate of at least 99%,
(d) deliver the medicament at an accuracy rate of at least 97% during delivery of a dose of the medicament having a volume of at least 200 milliliters, and/or
(e) deliver the medicament at an accuracy rate of at least 98% during delivery of a dose of the medicament having a volume of at least 250 milliliters.

22-25. (canceled)

26. A drug delivery device as in claim 18, wherein the at least one sensor includes an inlet pressure sensor positioned along the inlet fluid path and configured to measure inlet fluid pressure and an outlet pressure sensor positioned along the outlet fluid path and configured to measure outlet fluid pressure.

27. The drug delivery device of claim 18, further comprising:
a medicament container containing a medicament;
wherein the inlet fluid path is further configured to receive the medicament from the medicament container;
wherein the outlet fluid path is further configured to deliver the medicament to a patient.

28. The drug delivery device as in claim 27, wherein the medicament is in the form of a bispecific T cell engager (BiTE®), wherein the BiTE® is optionally a half-life extended (HLE) BiTE®.

29. (canceled)

30. The drug delivery device as in claim 27, wherein the controller includes an encoder-fed, closed loop system.

31-57. (canceled)
